SEQUENCE LISTING (2 sequences)

SEQ ID NO: 1 - DNA, 1567 bp, Homo sapiens
  source 1..1567: mol_type = genomic DNA; note = nucleotide sequence of RPN11
aagtgcttcgcggcggaggcccgggcaactcttttgaatggaatcgggctgattcatcgccggtttgcagacagagccgcgtcgggtgtgcgccgctgctgctgttgcctctgtcttcgcgtcaccacagaggcaagacaagggtccatatcgcggcatccggctcccgcccgtcttcaggagagaaagaaaaaataaaatatacttggggaagttgtacctgccagaattagcaagagctttctttaagaagacatttgtcaaactcaacaaattgaaggttaacaccttaagagttgtagttactgaccagaaatatggacagacttcttagacttggaggaggtatgcctggactgggccaggggccacctacagatgctcctgcagtggacacagcagaacaagtctatatctcttccctggcactgttaaaaatgttaaaacatggccgtgctggagttccaatggaagttatgggtttgatgcttggagaatttgttgatgattataccgtcagagtgattgatgtgtttgctatgccacagtcaggaacaggtgtcagtgtggaggcagttgatccagtgttccaagctaaaatgttggatatgttgaagcagacaggaaggccggagatggttgttggttggtatcacagtcaccctggctttggttgttggctttctggtgtggatatcaacactcagcagagctttgaagccttgtcggagagagctgtggcagtggttgtggatcccattcagagtgtaaaaggaaaggttgttattgatgccttcagattgatcaatgctaatatgatggtcttaggacatgaaccaagacaaacaacttcgaatctgggtcacttaaacaagccatctatccaggcattaattcatggactaaacagacattattactccattactattaactatcggaaaaatgaactggaacagaagatgttgctaaatttgcataagaagagttggatggaaggtttgacacttcaggactacagtgaacattgtaaacacaatgaatcagtggtaaaagagatgttggaattagccaagaattacaataaggctgtagaagaagaagataagatgacacctgaacagctggcaataaagaatgttggcaagcaggaccccaaacgtcatttggaggaacatgtggatgtacttatgacctcaaatattgtccagtgtttagcagctatgttggatactgtcgtatttaaataaagcaacgaaaaacgctattaatgatgccttcagtgtatattcctctgttgttcctaatgctcaaaatcaagggacctctgaaggtgtacttggctaaatgtaagacatctggcatcatttgcagcactgtaacaccttcagtctcagttgtgcaattacttctgtttctttagtcagggtctttgcagattctaaagttatacatgaatacatcaaagtggacaaattttgttaagatcccatttaatatttgaaaaaatcagtagcacaaatatattttgattgtcacttacaaaataaaatacatttacagtcta

SEQ ID NO: 2 - protein, 930 aa, Homo sapiens
  source 1..930: note = amino acid sequence of protein encoded by RPN11
METASPARGLEULEUARGLEUGLYGLYGLYMETPROGLYLEUGLYGLNGLYPROPROTHRASPALAPROALAVALASPTHRALAGLUGLNVALTYRILESERSERLEUALALEULEULYSMETLEULYSHISGLYARGALAGLYVALPROMETGLUVALMETGLYLEUMETLEUGLYGLUPHEVALASPASPTYRTHRVALARGVALILEASPVALPHEALAMETPROGLNSERGLYTHRGLYVALSERVALGLUALAVALASPPROVALPHEGLNALALYSMETLEUASPMETLEULYSGLNTHRGLYARGPROGLUMETVALVALGLYTRPTYRHISSERHISPROGLYPHEGLYCYSTRPLEUSERGLYVALASPILEASNTHRGLNGLNSERPHEGLUALALEUSERGLUARGALAVALALAVALVALVALASPPROILEGLNSERVALLYSGLYLYSVALVALILEASPALAPHEARGLEUILEASNALAASNMETMETVALLEUGLYHISGLUPROARGGLNTHRTHRSERASNLEUGLYHISLEUASNLYSPROSERILEGLNALALEUILEHISGLYLEUASNARGHISTYRTYRSERILETHRILEASNTYRARGLYSASNGLULEUGLUGLNLYSMETLEULEUASNLEUHISLYSLYSSERTRPMETGLUGLYLEUTHRLEUGLNASPTYRSERGLUHISCYSLYSHISASNGLUSERVALVALLYSGLUMETLEUGLULEUALALYSASNTYRASNLYSALAVALGLUGLUGLUASPLYSMETTHRPROGLUGLNLEUALAILELYSASNVALGLYLYSGLNASPPROLYSARGHISLEUGLUGLUHISVALASPVALLEUMETTHRSERASNILEVALGLNCYSLEUALAALAMETLEUASPTHRVALVALPHELYS

(19) United States
(12) Patent Application Publication
(10) Pub. No.: US 2025/0043360 A1
(43) Pub. Date: Feb. 6, 2025

(54) USE OF RPN11 MARKER IN DETECTION OF MYELOMA AND DISEASE RISK THEREOF, PROGNOSIS ANALYSIS AND TREATMENT MEDICAMENT

(71) Applicants: Peking University, Beijing (CN); Peking University People's Hospital, Beijing (CN)

(72) Inventors: Luyang Sun, Beijing (CN); Lin He, Beijing (CN); Shimiao Yu, Beijing (CN); Jin Lu, Beijing (CN); Xuelin Dou, Beijing (CN)

(21) Appl. No.: 18/717,404

(22) PCT Filed: Feb. 17, 2023
(86) PCT No.: PCT/CN2023/076683

§ 371 Date: Jun. 6, 2024

(30) Foreign Application Priority Data
Jun. 7, 2022 (CN) 202210636398.2

(51) Int. Cl. C12Q 1/6886; A61K 31/4709; A61P 35/00; G01N 33/574; G01N 33/68

(52) U.S. Cl. CPC C12Q 1/6886; A61K 31/4709; A61P 35/00; G01N 33/57407; G01N 33/6893; C12Q 2600/106; C12Q 2600/112; C12Q 2600/158; G01N 2333/4703

(57) ABSTRACT

The present disclosure provides the utilization of an RPN11 marker in detecting myeloma and assessing disease risk, prognostic analysis, and therapeutic drug development. This includes a detection reagent and a kit for detecting smoldering multiple myeloma (SMM) and multiple myeloma (MM) and assessing disease risk. Additionally, it involves molecular typing of MM patients' cassette, prognostic analysis of the MM patients, and predicting the response of the MM patients to a bortezomib treatment. It entails the use of an RPN11 inhibitor in preparation of a pharmaceutical composition for treating MM characterized by drug-resistant t(4;14) translocation and a high RPN11 expression level. Notably, the combined use of the RPN11 with histone methyltransferase (HMT) enhances detection sensitivity and accuracy, preventing a mismatch between the patients' genotype and the prediction of disease development, and can effectively predict the overall survival and prognosis of MM patients with t(4;14) translocation.

### CROSS-REFERENCE TO RELATED APPLICATIONS

This patent application is a national stage application of International Patent Application No. PCT/CN2023/076683, filed on Feb. 17, 2023, which claims the benefit and priority of Chinese Patent Application No. 202210636398.2 filed on Jun. 7, 2022, the contents of both of which are incorporated herein by reference in their entirety.

### REFERENCE TO SEQUENCE LISTING

A computer readable XML file entitled “GWPCT20240301888_sequence listing.xml”, which was created on Apr. 7, 2024, with a file size of about 5,530 bytes, contains the sequence listing for this application, has been filed with this application and is hereby incorporated by reference in its entirety.

### TECHNICAL FIELD OF THE INVENTION

The present disclosure belongs to the field of diagnosis and treatment of multiple myeloma (MM) and particularly relates to the use of an RPN11 marker in detecting myeloma and a disease risk, molecular typing, a prognostic analysis, and a therapeutic drug thereof.

### BACKGROUND OF THE INVENTION

Multiple myeloma (MM) is the second largest hematological malignant tumor after lymphoma. The main clinical feature of MM is a clonal malignant proliferative disease of plasma cells in the blood and bone marrow, accounting for about 10% of hematological malignancies. A “2020 Cancer Data Report” published by the American Cancer Society in 2021 showed that the annual new cases of MM were 176,404, accounting for 0.9% of all new tumor cases; there were 117,077 new deaths in the year, accounting for 1.2% of all new cancer deaths. The current incidence of MM in China is also showing an increasing trend year by year, and the median age of onset in China is significantly earlier than that in Western countries compared with that in European and American countries. Current models suggest that MM progresses from monoclonal gammopathy of unknown significance (MGUS) to smoldering multiple myeloma (SMM), to multiple myeloma (MM) that is symptomatic, and ultimately to plasma cell leukemia (PML).

MM is a highly heterogeneous malignant plasma cell disease. In the past few years, with the emergence of new drugs, the survival of MM patients has been greatly improved, but there are still challenges in molecular typing and prognosis assessment. Existing research shows that almost all MM cases have cytogenetic abnormalities, manifested as enhancer translocation of the IgH gene to form fusion genes, hyperdiploidy, and subdiploid karyotypes, mainly translocation of the IgH gene enhancer located on chromosome 14. The IgH gene enhancer can be translocated and fused with different chromosomes, among which t(4;14) is a high-risk genotype, accounting for about 15% to 20% of patients. t(4;14) is the enhancer fusion of the chromosome 4 gene FGFR/MMSET and the IgH gene. Abnormally high expression levels of histone methyltransferase (HMT) (specifically MM Suppressor of variegation, Enhancer of zeste, and Trithorax, MMSET) can occur in such patients. At present, the treatment plan and prognosis assessment of MM patients are determined mainly relying on genotyping of IgH gene enhancer translocation, but about 20% of patients have a mismatch between genotype and disease progression prediction. Currently, there is a lack of a type of marker that can effectively predict the overall survival and prognosis of MM patients with t(4;14) translocation. At the same time, there is also a lack of a molecular typing scheme that is independent of IgH gene enhancer translocation and can effectively indicate prognosis and treatment options.

In addition, inhibitors targeting the 20S proteasome, represented by bortezomib, have significantly improved the overall survival rate of MM patients in recent years. However, proteasome inhibitor drugs can still cause serious side effects such as thrombocytopenia, gastrointestinal disorders, and peripheral neuropathy during treatment. Overcoming the toxicity and resistance of bortezomib therapy is a difficult problem in the treatment of MM patients, especially for patients with t(4;14) translocation MM. Moreover, there is currently a lack of markers that can effectively predict the response of MM patients to bortezomib treatment.

### BRIEF SUMMARY OF THE INVENTION

An objective of the present disclosure is to provide a detection reagent and kit for detecting SMM and MM and a disease risk thereof, molecular typing of an MM patient, a prognostic analysis of the MM patient(especially with t(4;14) translocation), and predicting a response of the MM patient to a bortezomib treatment, and to provide a novel pharmaceutical composition for the treatment of t(4;14) translocation MM. In the present disclosure, “RPN11” refers to a protein encoded by the nucleotide sequence corresponding to Gene ID No. 10213 and a protein shown in the corresponding coding sequence NP_005796.1, as well as a protein purified in prokaryotic and eukaryotic systems. RPN11 has the nucleotide sequence set forth in SEQ ID NO: 1 and an encoded protein sequence set forth in SEQ ID NO: 2.

In order to achieve the objective of the present disclosure, during the research, the present disclosure provides an influence of an RPN11 gene and an encoded protein thereof on the survival and proliferation of tumor cells (with t(4;14) translocation) in MM, including:
1) an RPN11 protein purified in vitro from a prokaryotic system is provided;
2) an shRNA sequence of the RPN11 gene is provided;
3) an MM LP-1 cell line (with t(4;14) translocation) that stably overexpresses or knocks down the RPN11 gene using lentivirus is provided; and
4) an MM animal model formed by injecting NOG mice (NOD/Shi-scid IL2rγnull mice) with KMS11 cells stably overexpressing or knocking down the RPN11 gene (a cell line with t(4;14) translocation) is provided.

In view of this, the present disclosure provides the following solutions.

A first aspect of the present disclosure provides a detection reagent for detecting SMM and MM (especially with t(4;14) translocation) and a disease risk thereof, molecular typing of an MM patient, prognostic analysis of the MM patient, and predicting a response of a bortezomib treatment, including the following components:
(1) an RPN11-specific binding molecule and/or an RPN11-specific antibody; and
(2) a primer or a primer pair, a probe, or a chip that specifically amplifies an RPN11 mRNA or an RPN11 cDNA.

Preferably, the detection reagent includes an MMSET-specific antibody and/or an MMSET-specific binding molecule.

Preferably, the detection reagent includes a primer or a primer pair, a probe, or a chip that specifically amplifies an MMSET mRNA or an MMSET cDNA.

In Examples 1 to 4, the prognostic analysis includes analyzing the RPN11 expression level and the MMSET expression level individually or jointly to evaluate the prognosis of MM patients.

In Examples 1 to 4, the prognostic analysis evaluation includes predicting the survival time of MM patients receiving chemotherapy or targeted therapy.

In Examples 1 to 4, the protein includes a full-length protein or a protein fragment.

In Examples 1 to 4, the detection reagent includes an RPN11-specific antibody, an RPN11-specific binding molecule, a specific amplification primer, a probe, a nucleic acid chip (such as a DNA chip), or a protein chip.

In Examples 1 to 4, the detection reagent is coupled to or carries a detectable label.

In Examples 1 to 4, the detectable label is selected from the group consisting of a chromophore, a chemiluminescent group, a fluorophore, an isotope, and an enzyme.

In Examples 1 to 4, the nucleic acid chip includes a substrate and a specific oligonucleotide probe spotted on the substrate; and the specific oligonucleotide probe includes a probe that specifically binds to an RPN11 polynucleotide (mRNA or DNA).

In Examples 1 to 4, the protein chip includes a substrate and a specific antibody spotted on the substrate, and the specific antibody includes an anti-RPN11 specific antibody.

In Examples 1 to 4, the in vitro sample is one or more selected from the group consisting of a tissue sample, a cell sample, and a blood sample.

In Examples 1 to 4, the cell sample includes a cell sample from an MGUS patient, a cell sample from an SMM patient, a cell sample from an MM patient, and a cell sample from a normal volunteer.

In Examples 1 to 4, the cell sample includes a cell sample from a treatment-naive MM patient, a cell sample from a bortezomib-relapsed or resistant MM patient, and a cell sample from a normal volunteer.

In Examples 1 to 4, the cell sample includes a cell sample from MM patients with low RPN11 expression/low MMSET expression, a cell sample from MM patients with low RPN11 expression/high MMSET expression, a cell sample from MM patients with high RPN11 expression/low MMSET expression, and a cell sample from MM patients with high RPN11 expression/high MMSET expression. The high expression of MMSET is an important feature of MM patients with t(4;14) translocation. In the present disclosure, the expressions of RPN11 and MMSET are combined for the first time to accurately predict the prognostic characteristics of MM patients with t(4;14) translocation.

A second aspect of the present disclosure is to design a kit for detecting SMM and MM (especially with t(4;14) translocation) and a disease risk thereof, molecular typing of an MM patient, a prognostic analysis of the MM patient, and predicting a response of the MM patient to a bortezomib treatment, where the kit includes one or a combination of two or more selected from the group consisting of an RPN11 detection gene, an RPN11 mRNA, an RPN11 cDNA, and an RPN11 expression protein.

Further, the kit includes one or more selected from the group consisting of an MMSET gene, an MMSET mRNA, an MMSET cDNA, and an MMSET expression protein.

Further, the kit further includes one or more selected from the group consisting of an RPN11 gene, an RPN11 mRNA, an RPN11 cDNA, and an RPN11 expression protein to serve as a reference substance or a quality control.

Further, the kit further includes one or more selected from the group consisting of an MMSET gene, an MMSET mRNA, an MMSET cDNA, and an MMSET expression protein to serve as a reference substance or a quality control.

In Examples 1 to 4, the kit further includes a label or instruction, and the label or instruction indicates that the kit is used to detect SMM or MM and its disease risk.

In Examples 1 to 4, the kit further includes a gene, an mRNA, a cDNA, and/or a protein of the RPN11 (or also MMSET) as a reference substance or a quality control.

In Examples 1 to 4, the detection of SMM or MM and its disease risk refers to a process for detecting whether it has occurred and/or determining the possibility (susceptibility) of occurrence.

In Examples 1 to 4, the determination includes pre-determining (predicting) the prognosis of MM patients and the response of patients of this subtype to bortezomib treatment.

In Examples 1 to 4, when the kit is used to detect SMM or MM or its disease risk and to predict the prognosis of MM patients or the response to bortezomib treatment, the label or instruction indicates the following: if the RPN11 expression level of a tested subject is significantly higher than a control expression level, the risk of the tested subject suffering from MM or having poor response to bortezomib treatment is greater than that of normal people.

In Examples 1 to 4, the control expression level is an expression level of RPN11 in a same sample in the normal population, and an expression level of RPN11 in the same sample in the general cancer patient population except for MM patients.

A third aspect of the present disclosure provides the use of an RPN11 inhibitor in preparation of a pharmaceutical composition for treating MM with drug-resistant t(4;14) translocation and a high RPN11 expression level.

Further, the pharmaceutical composition includes an RPN11 inhibitor and lenalidomide or dexamethasone.

Further, the RPN11 inhibitor is one more selected from the group consisting of a neutralizing antibody, a small-molecule compound, and an antisense nucleic acid.

Further, the RPN11 inhibitor is a small-molecule inhibitor Capzimin.

In Examples 5 to 8, the RPN11 inhibitor is one or more selected from the group consisting of a neutralizing antibody, a small-molecule compound, and an antisense nucleic acid.

In Examples 5 to 8, the RPN11 inhibitor is used for MM patients with t(4;14) translocation who are resistant to proteasome inhibitor-targeted therapy.

In Examples 5 to 8, the RPN11 inhibitor is used alone, or in combination with the proteasome inhibitor, or in combination with a sensitizing immune modulator or an anti-tumor auxiliary drug.

In particular, the RPN11 inhibitor is the small-molecule inhibitor Capzimin, which shows smaller dosage, more significant therapeutic effect, and less toxic and milder side effects than those of known RPN11 inhibitors (such as OPA). For the first time, OPA and Capzimin are used in t(4;14) translocation MM, and their synergistic sensitizing effect on traditional therapy of t(4;14) translocation MM is found.

In Examples 5 to 8, the component accounts for 0.1 wt % to 99.9 wt %, preferably 10 wt % to 99.9 wt %, and more preferably 70% to 99.9 wt % of the total weight of the pharmaceutical composition.

In Examples 5 to 8, the pharmaceutical composition is a liquid, solid, or semi-solid.

In Examples 5 to 8, a dosage form of the pharmaceutical composition is an oral preparation, injection, or external pharmaceutical agent.

In Examples 5 to 8, the carrier includes an infusion carrier and/or an injection carrier; preferably, the carrier is one or more selected from the group consisting of physiological saline, glucose saline, and a combination thereof.

The beneficial effects produced by adopting the above technical solutions are as follows:
1. In the present disclosure, the expression of RPN11 gradually increases with the progression of MM (MUGS, SMM, and MM). Meanwhile, MM patients with high expression levels of RPN11 and MMSET have a worse prognosis, and MM patients with high expression level of RPN11 have a worse response to bortezomib treatment. Based on this, the designed detection reagent and kit for detecting the SMM and the MM and the disease risk thereof, the molecular typing of the MM patient, the prognostic analysis of the MM patient, and predicting the response of the MM patient to the bortezomib treatment has more sensitive and accurate detection performance. In particular, combined use of the RPN11 with the HMT (specifically MMSET) avoids a mismatch between the patient's genotype and the prediction of disease development, and can effectively predict the overall survival and prognosis of MM patients with t(4;14) translocation.
2. In the novel pharmaceutical composition provided by the present disclosure for the treatment of t(4;14) translocation MM, the RPN11 inhibitor can effectively promote the apoptosis of t(4;14) translocation MM cells.
3. In the present disclosure, the RPN11 inhibitor Capzimin can overcome bortezomib resistance and enhance the synergistic therapeutic effect of lenalidomide and dexamethasone in t(4;14) translocation MM. The present disclosure provides a new solution for personalized treatment of t(4;14) translocation MM.

### BRIEF DESCRIPTION OF THE DRAWINGS

FIG. 1 shows the expression of RPN11 in MGUS, SMM, and MM provided in an example of the present disclosure; where * represents p<0.05, ** represents p<0.01, and *** represents p<0.001;

FIG. 2 shows the relationship between RPN11 expression level and prognosis provided in an example of the present disclosure;

FIG. 3 shows the relationship between conjoint analysis of RPN11 expression level and MMSET expression level and prognosis;

FIG. 4A-FIG. 4D show the relationship between RPN11 expression level and bortezomib treatment response;

FIG. 5A-FIG. 5C show the influence of RPN11 on the cell phenotype of t(4;14) translocation MM;

FIG. 6A-FIG. 6B show the results of subcutaneous tumor formation experiments in the animal phenotype: NOG mice (NOD/Shi-scid IL2rγnull mice);

FIG. 7A-FIG. 7B show the inhibitory effect of RPN11 inhibitor (OPA or Capzimin) on t(4;14) translocation MM cells; and

FIG. 8A-FIG. 8D show the synergistic effect of RPN11 inhibitor (OPA or Capzimin) with Lenalidomide or Dexamethasone.

### DETAILED DESCRIPTION OF THE EMBODIMENTS

The present disclosure is further described below with reference to examples.

### Example 1

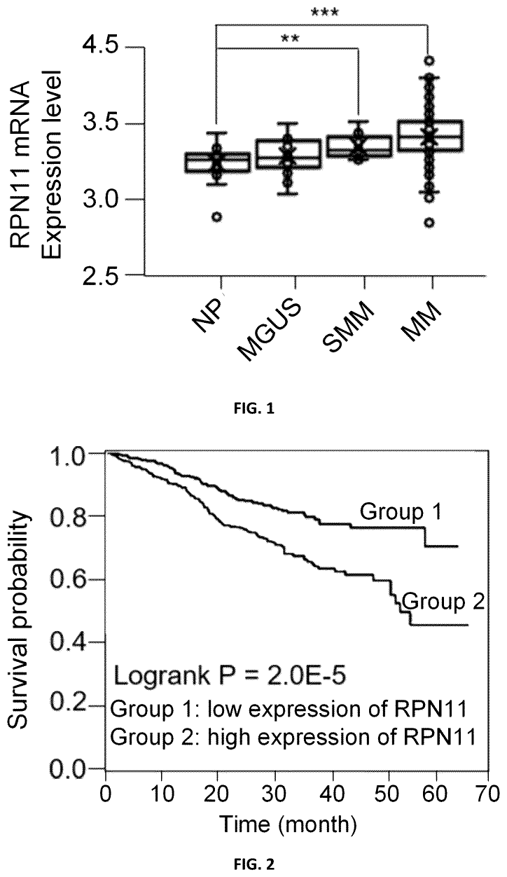

The expression levels of RPN11 gene in normal healthy people (n=22), patient samples MGUS (n=44), SMM (n=12), and MM (n=538) were obtained from GSE5900 and GSE4204 numerical aggregation. The results are shown in FIG. 1. Compared with normal healthy samples, RPN11 expression levels were significantly increased in SMM and MM patient samples.

### Example 2

Kaplan-Meier survival analysis was conducted on a large-scale MM patient sample (n=858) data set(Multiple Myeloma Research Foundation). The results are shown in FIG. 2. Higher RPN11 expression levels in MM patients were significantly associated with shorter overall survival. Higher expression of RPN11 in MM patients led to a worse prognosis.

### Example 3

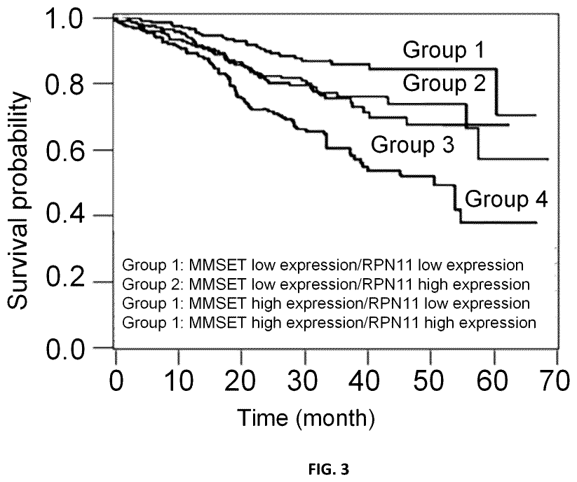

Kaplan-Meier survival analysis was conducted on a large-scale MM patient sample (n=858) data set(Multiple Myeloma Research Foundation). The results are shown in FIG. 3. MM patients with high expression of both RPN11 and MMSET had the worst prognosis. This indicated that in MM patients with high MMSET expression, high RPN11 expression could be used as an effective indicator to improve the accuracy of predicting the patient's overall survival and overall survival rate. Since MMSET was highly expressed in MM patients with t(4;14) translocation, this example also demonstrated that high expression of RPN11 was an effective indicator for predicting the prognosis of MM patients with t(4;14) translocation.

### Example 4

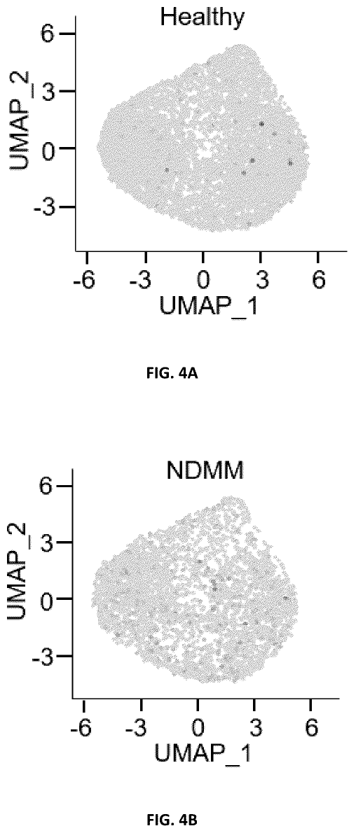

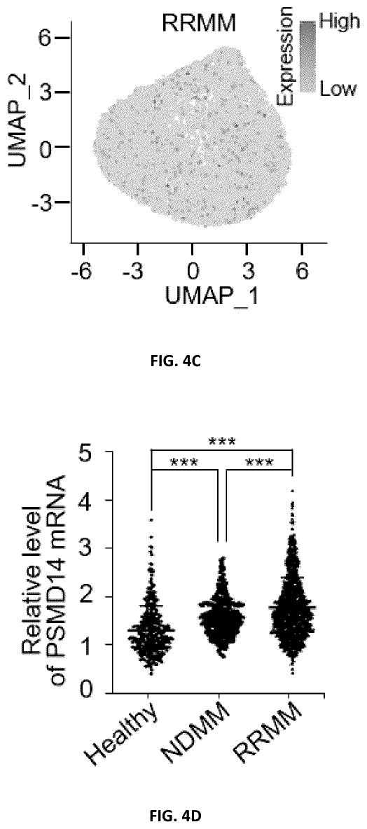

The expression levels of the RPN11 gene in MM cells (CD138 positive) of the normal healthy subjects (n=22), treatment-naive MM patients (NDMM, n=9), and bortezomib-relapsed or resistant MM patients (early progression after bortezomib treatment(less than 18 months) or failure to respond effectively to initial bortezomib treatment(more than 4 months), RRMM, n=35) samples were obtained from the single-cell sequencing number aggregation of GSE161195 and GSE117156. The results are shown in FIG. 4A-FIG. 4D. Compared with normal healthy people (healthy), RPN11 expression levels were significantly increased in samples from NDMM and RRMM patients; while compared with NDMM, RPN11 expression levels were also significantly increased in RRMM patients. These indicated that high expression of RPN11 could be used as an effective indicator to predict patients' response to bortezomib treatment.

### Example 5

In this example, the LP-1 cell line used was a t(4;14) translocation MM cell line to specifically illustrate the effect of RPN11 on the phenotype of t(4;14) translocation MM cells.

Construction of LP-1 cell line that stably overexpressed and knocked down RPN11: the cDNA sequence of RPN11 was cloned into a GV208 expression plasmid, and the shRNA sequence targeting RPN11 was cloned into GV112. An obtained recombinant was co-transfected into HEK-293T cells with an auxiliary vector. After 48 h, the virus supernatant was collected, filtered, clarified, and concentrated by ultracentrifugation, and a concentrated virus was used to infect LP-1 cells with HiTransB-2.

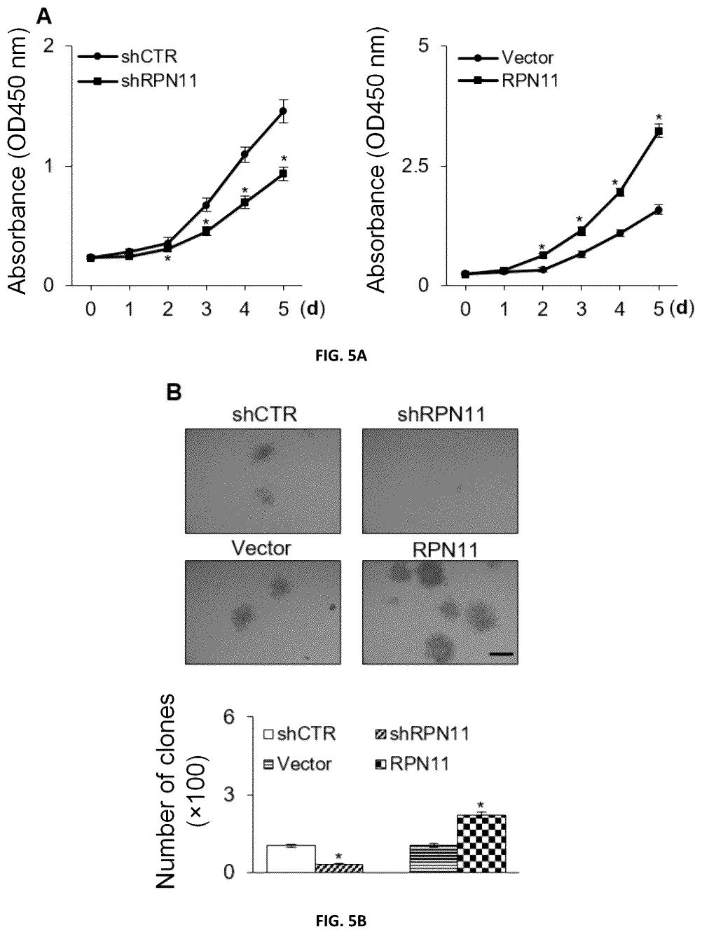

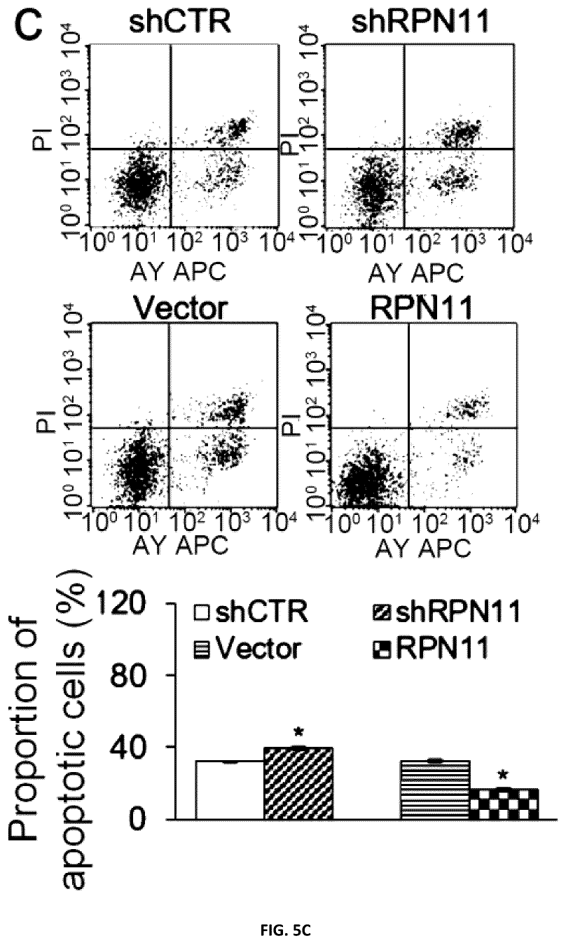

CCK kit was used to detect cell proliferation of LP-1 cells stably overexpressing and knocking down RPN11: after the transfected cells were cultured for 48 h, the cells were digested with 0.25% trypsin and counted, inoculated in a 96-well plate at a concentration of 1×104 cells/mL, 100 μL in each well; 10 μL of CCK8 reagent was added to each well of the 96-well plate in the dark, the culture plate was shaken for 3 min, then placed into the incubator to continue culturing for 2 h, and the culture plate was taken out and the OD value was measured at a wavelength of 450 nm with a microplate reader. As shown in FIG. 5A, after knocking down RPN11, cell proliferation was significantly inhibited compared with the control group; after overexpression of RPN11, cell proliferation ability was significantly enhanced. Therefore, RPN11 could significantly promote the proliferation of t(4;14) translocation MM cells.

The clonogenic ability of LP-1 cells stably overexpressing and knocking down RPN11 was analyzed. The cells were counted and transferred to a 6-well plate, with 5,000 cells per well, and cultured in a 37° C. incubator for 14 d. The medium was changed every two days, the cells were washed 2 times with cold PBS and fixated with 4% paraformaldehyde at room temperature for 10 min, stained with 0.1% crystal violet staining solution for 20 min, and rinsed with PBS. As shown in FIG. 5B, after knocking down RPN11, cell colony formation was significantly inhibited compared with the control group; after overexpression of RPN11, cell colony formation was significantly enhanced. Therefore, RPN11 could significantly promote the colony formation of t(4;14) translocation MM cells.

Apoptosis was detected in LP-1 cells stably overexpressing and knocking down RPN11. The cells were treated with camptothecin (CPT) for 24 h, washed with PBS, double-stained with Annexin V-APC/propidium iodide (PI) for 15 min at room temperature in the dark, and cell collection and analysis were conducted on each sample using a FACS Calibur flow cytometer. As shown in FIG. 5C, after knocking down RPN11, cell apoptosis was significantly increased compared with the control group; after overexpression of RPN11, cell apoptosis was significantly reduced. Therefore, RPN11 could significantly inhibit the apoptosis of t(4;14) translocation MM cells.

### Example 6

In this example, the KMS11 cell line used was an MM cell line containing the t(4;14) translocation to specifically illustrate the influence of RPN11 on the phenotype of MM animals with the t(4;14) translocation.

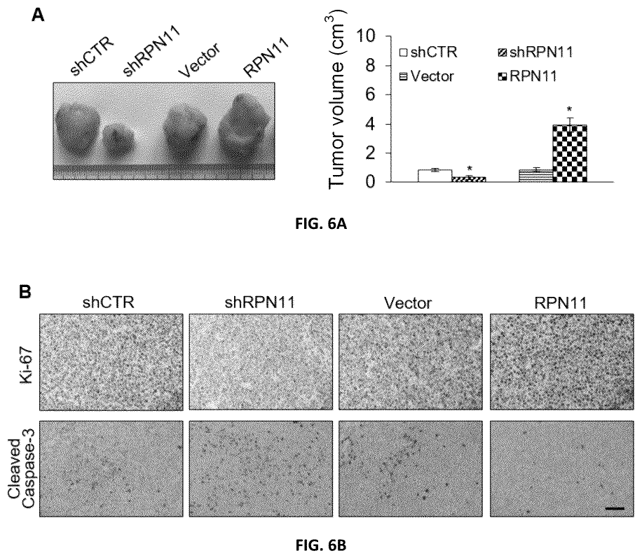

KMS11 cells were infected with lentivirus that overexpressed or knocked down RPN11 for tumor xenograft experiments, and KMS11 cells (5×10⁶ cells) were subcutaneously injected into 5-week-old female NOG mice. The proliferation and apoptosis levels of transplanted tumors were detected by tissue immunochemical staining for Ki-67 and Cleaved caspase-3. As shown in FIG. 6A-FIG. 6B, after knocking down RPN11, the proliferation ability of transplanted tumors was reduced while the number of apoptotic cells was significantly increased; after overexpression of RPN11, the proliferation ability of transplanted tumors was increased while the number of apoptotic cells was significantly reduced.

### Example 7

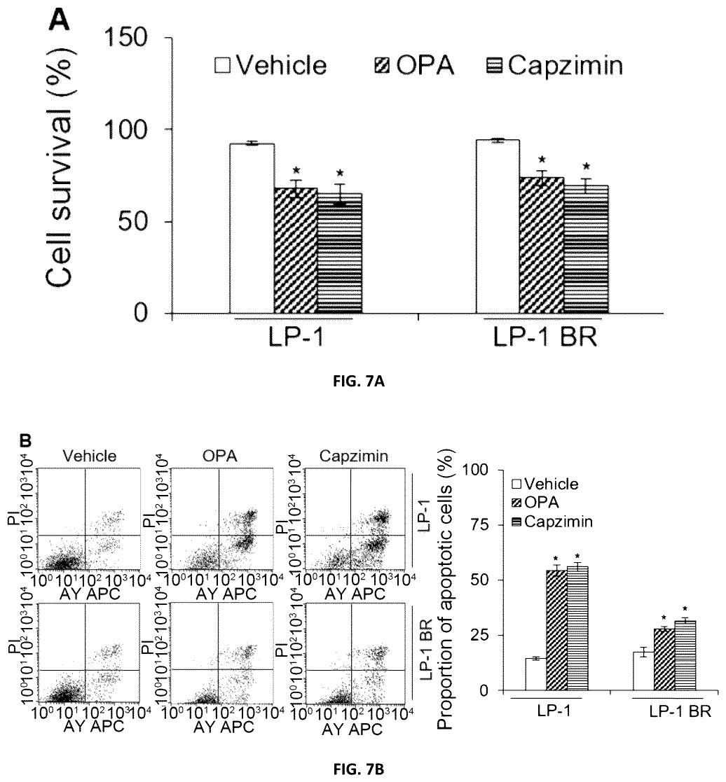

Wild-type or bortezomib-resistant LP-1 cells (LP-1 or LP-1BR) were treated with Vehicle or RPN11 inhibitor (OPA or Capzimin) separately, and the proliferation and apoptosis of LP-1BR cells were detected through CCK cell counting experiment and cell apoptosis experiment. As shown in FIG. 7A-FIG. 7B, compared with the control group, the proliferation ability of LP-1 and LP-1BR cells treated with RPN11 inhibitor was significantly reduced and the number of apoptotic cells was significantly increased. CCK cell counting experiment showed that cell viability was weakened (FIG. 7A), and the apoptosis experiment showed that the RPN11 inhibitor could promote the apoptosis of LP-1 and LP-1BR cells (FIG. 7B). Moreover, compared with the traditional inhibitor OPA (concentration used in vitro at 10 μM), the novel RPN11 inhibitor Capzimin (concentration used in vitro at 2 μM) had a more significant effect in inhibiting the survival of LP-1 and LP-1BR cells and promoting the apoptosis of LP-1 and LP-1BR cells, while a dosage of the drug was lower.

### Example 8

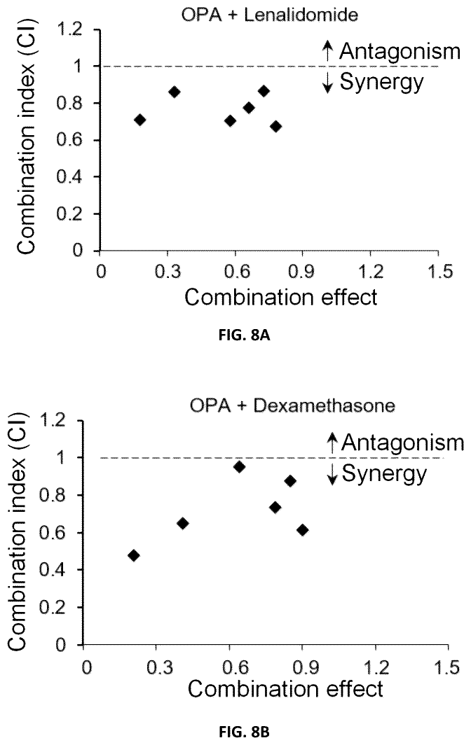

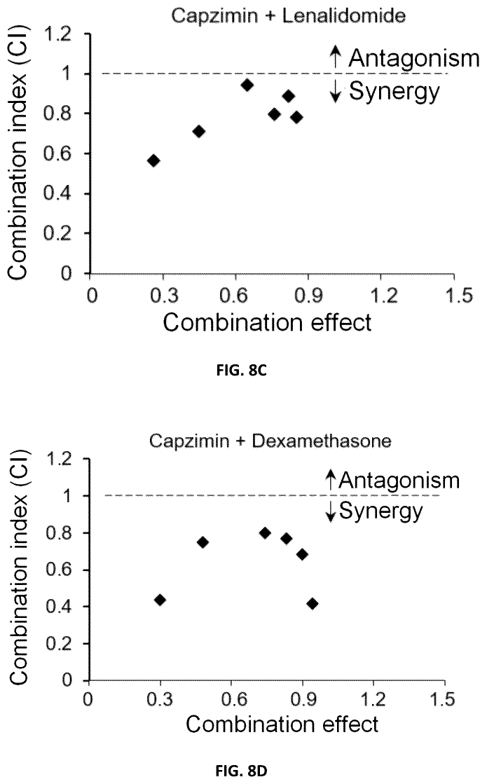

LP-1 cells were treated with RPN11 inhibitors (OPA or Capzimin) or/and Lenalidomide or Dexamethasone (within a range of concentrations), and the cell proliferation ability was detected using CCK cell counting assay. As shown in FIG. 8A-FIG. 8D, compared with RPN11 inhibitor or lenalidomide or dexamethasone alone, the combined treatment of RPN11 inhibitor and lenalidomide/dexamethasone showed a synergistic effect on inhibiting the growth and proliferation of LP-1 cells. Moreover, compared with the traditional inhibitor OPA, the novel RPN11 inhibitor Capzimin, used in conjunction with lenalidomide or dexamethasone, had a more significant inhibitory effect on the proliferation of LP-1 cells, while a dosage of the drug was lower.

The above embodiment is illustration and description of the present disclosure for ease of understanding, rather than any limit to the present disclosure. Any modification, equivalent substitution, improvement, and the like made within the spirit and principle of the present disclosure shall fall within the protection scope of the present disclosure. The above are merely preferred specific embodiments of the present disclosure, but the protection scope of the present disclosure is not limited thereto. Any equivalent replacement or modification made by a person skilled in the art according to the technical solutions of the present disclosure and inventive concepts thereof within the technical scope of the present disclosure shall fall within the protection scope of the present disclosure. Finally, it should be noted that the foregoing embodiments are only intended to describe, rather than to limit the technical solutions of the present disclosure. Although the present disclosure is described in detail with reference to the preferred embodiments, those of ordinary skill in the art should understand that modifications or equivalent replacements may be made to the technical solutions of the present disclosure without departing from the spirit and scope of the technical solutions of the present disclosure.

1. A detection reagent for detection of smoldering multiple myeloma (SMM) and multiple myeloma (MM) and a disease risk thereof, molecular typing of an MM patient, prognostic analysis of the MM patient, and prediction of a response of the MM patient to bortezomib treatment, comprising the following components:
(1) an RPN11-specific binding molecule and/or an RPN11-specific antibody; and
(2) a primer or a primer pair, a probe, or a chip that specifically amplifies an RPN11 mRNA or an RPN11 cDNA.

2. The detection reagent according to claim 1, wherein the detection reagent comprises an MM Suppressor of variegation, Enhancer of zeste, and Trithorax (MMSET)-specific antibody and/or an MMSET-specific binding molecule.

3. The detection reagent according to claim 1, wherein the detection reagent comprises a primer or a primer pair, a probe, or a chip that specifically amplifies an MMSET mRNA or an MMSET cDNA.

4. A detection kit for detection of SMM and MM and a disease risk thereof, molecular typing of an MM patient, prognostic analysis of the MM patient, and prediction of a response of the MM patient to a bortezomib treatment, wherein the kit comprises at least one selected from the group consisting of an RPN11 detection gene, an RPN11 mRNA, an RPN11 cDNA, and an RPN11 expression protein.

5. The detection kit according to claim 4, wherein the kit comprises at least one selected from the group consisting of an MMSET gene, an MMSET mRNA, an MMSET cDNA, and an MMSET expression protein.

6. The detection kit according to claim 4, wherein the kit further comprises at least one selected from the group consisting of an RPN11 gene, an RPN11 mRNA, an RPN11 cDNA, and an RPN11 expression protein to serve as a reference substance or a quality control.

7. The detection kit according to claim 4, wherein the kit further comprises at least one selected from the group consisting of an MMSET gene, an MMSET mRNA, an MMSET cDNA, and an MMSET expression protein to serve as a reference substance or a quality control.

8. A method for treating MM with drug-resistant t(4;14) translocation and a high RPN11 expression level in a subject in need thereof, comprising administrating an effective amount of RPN11 inhibitor to the subject.

9. The method according to claim 8, wherein the RPN11 inhibitor is at least one selected from the group consisting of a neutralizing antibody, a small-molecule compound, and an antisense nucleic acid.

10. The method according to claim 8, wherein the RPN11 inhibitor is a small-molecule inhibitor Capzimin.
